(19) United States
(12) Patent Application Publication
(10) Pub. No.: US 2022/0038613 A1
(43) Pub. Date: Feb. 3, 2022

(54) IMAGING DEVICE

(71) Applicant: OLYMPUS CORPORATION, Tokyo (JP)

(72) Inventors: Takashi KOBAYASHI, Tokyo (JP); Satoru ADACHI, Tsuchiura-shi (JP); Makoto IKEDA, Tokyo (JP)

(73) Assignee: OLYMPUS CORPORATION, Tokyo (JP)

(21) Appl. No.: 17/379,330

(22) Filed: Jul. 19, 2021

Related U.S. Application Data
(60) Provisional application No. 63/058,524, filed on Jul. 30, 2020.

(51) Int. Cl. H04N 5/235; H04N 5/225; H04N 9/04; A61B 1/05; A61B 1/06

(52) U.S. Cl. CPC H04N 5/2352; H04N 5/2256; H04N 2005/2255; A61B 1/05; A61B 1/0638; H04N 9/0451

(57) ABSTRACT

An imaging device for performing phase-difference detection auto-focus includes an image sensor including a plurality of normal pixels and a plurality of phase-difference detection pixels arranged in a matrix shape; an exposure control circuit controlling a first exposure time driving the normal pixels for generating image signals, and a second exposure time driving the phase-difference detection pixels for generating phase-difference detection signals; and a lighting device irradiating the imaging object with a first irradiating light at the first exposure time, and irradiating the imaging object with a second irradiating light at the second exposure time. The wave length range of the second irradiating light is narrower than the wave length range of the first irradiating light.

### RELATED APPLICATION DATA

This application is based on and claims priority under 35 U.S.C. § 119 to U.S. Provisional Application No. 63/058,524, filed Jul. 30, 2020, the entire contents of which are incorporated herein by reference.

### FIELD OF THE INVENTION

The present disclosure relates to an imaging device, and more particularly, to an imaging device performing phase-difference detection auto-focus (PDAF), a method of performing the PDAF, and a program performing the same.

### DESCRIPTION OF THE RELATED ART

An imaging device uses auto-focus (AF) measurement to ensure that pictures taken by the user have improved sharpness regardless of subject distance. Such an imaging device may be used in medical equipment, digital (video/still) cameras, or the like. The AF measurements include a PDAF, a contrast detection AF (hereinafter, also referred to as “CDAF”), and a hybrid AF, in which the PDAF and the CDAF are combined.

In an imaging device that uses the PDAF technology, AF measurement is performed by measuring a phase-difference between two images obtained by splitting an exit pupil of an imaging optical system. To perform the PDAF measurement, an external phase-difference detection sensor is provided in addition to an image sensor serving as an imaging element for taking an image, and a phase-difference is obtained on the basis of output of the phase-difference detection sensor.

The PDAF measurement is advantageous in that AF scanning operation for moving a focus position (lens position) of the imaging optical system does not need to be performed at the time of AF, and therefore it is possible to execute AF for a comparatively short time. Thus, the PDAF measurement is excellent in terms of execution time of AF (detection time of the focusing position). However, the conventional PDAF measurement is disadvantageous in terms of accuracy of AF (detection accuracy of the focusing position). For example, in a case where the PDAF measurement is applied at the time of capturing an image, in particular, a moving image or continuously taking images, focusing accuracy is reduced.

This problem of the conventional PDAF measurement relates to circle of confusion (CoC). The CoC is the measurement of the optical blur circles created by the lens when light converges inside an imaging device. Light convergences that do not fall exactly on a focal plane create circles of light or blur spots. These blur spots become bigger as the light converges further from or nearer to the focal plane. Smaller circles mean it is more in-focus and sharp, while bigger blur spots are less in-focus and unsharp. The conventional PDAF measurement is performed by irradiating white light, which has a wavelength range of 400 nm-830 nm, the white light passes through the lens and is divided into different color lights with different wavelengths (R, G, B). When these different color lights with different wavelengths converge inside the imaging device, the convergences of the different color lights form different circles having different diameters, thereby causing bigger blur spots around the focal plane. As a result, the accuracy of the PDAF measurement is impaired, and becomes more difficult to obtain when the pixel size of the image sensor gets smaller.

### SUMMARY OF THE INVENTION

Accordingly, the present disclosure is directed to an imaging device that substantially obviates one or more of the issues due to limitations and disadvantages of related art endoscope.

An object of the present disclosure is to provide an imaging device for performing PDAF, which comprises an image sensor including a plurality of normal pixels and a plurality of phase-difference detection pixels arranged in a matrix shape; an exposure control circuit controlling a first exposure time driving the normal pixels for generating image signals, and a second exposure time driving the phase-difference detection pixels for generating phase-difference detection signals; and a lighting device irradiating the imaging object with a first irradiating light at the first exposure time, and irradiating the imaging object with a second irradiating light at the second exposure time, wherein a wave length range of the second irradiating light is narrower than a wave length range of the first irradiating light.

Another object of the present disclosure is to provide a method of performing PDAF for an imaging device including an image sensor. The method comprises arranging, in the image sensor, a plurality of normal pixels and a plurality of phase-difference detection pixels in a matrix shape; controlling, by a exposure controlling circuit arranged the image sensor, a first exposure time driving the normal pixels for generating image signals, and a second exposure time driving the phase-difference detection pixels for generating phase-difference detection signals; and irradiating, by a lighting source, an object to be imaged with a first irradiating light at the first exposure time, and irradiating the object with a second irradiating light at the second exposure time, wherein a wave length range of the second irradiating light is narrower than a wave length range of the first irradiating light.

Still another object of the present disclosure is to provide a non-transitory computer-readable medium in an electronic device that includes a built-in image sensor, a lighting source, a processor, and memory for storing programs to be executed by the processor, comprising instructions of identifying relevant information of an object, performs operations comprising controlling a first exposure time driving normal pixels of the image sensor for generating image signals of an object to be imaged, and a second exposure time driving phase-difference detection pixels of the image sensor for generating phase-difference detection signals; and irradiating, by the lighting source, the object to be imaged with a first irradiating light at the first exposure time, and irradiating the object with a second irradiating light at the second exposure time, wherein a wave length range of the second irradiating light is narrower than a wave length range of the first irradiating light.

Still another object of the present disclosure is to provide an endoscope system, comprising an endoscope including a tip portion to be inserted into an object; an imaging device arranged inside the tip portion; and a lighting device connected to the endoscope and providing illumination for the imaging device to capture an image of a region of the object to be examined based on the PDAF. The imaging device includes an image sensor including a plurality of normal pixels and a plurality of phase-difference detection pixels arranged in a matrix shape; an exposure control circuit controlling a first exposure time driving the normal pixels for generating image signals, and a second exposure time driving the phase-difference detection pixels for generating phase-difference detection signals; and a lighting device irradiating the imaging object with a first irradiating light at the first exposure time, and irradiating the imaging object with a second irradiating light at the second exposure time, wherein a wave length range of the second irradiating light is narrower than a wave length range of the first irradiating light.

Additional features and advantages will be set forth in the description that follows, and in part will be apparent from the description, or may be learned by practice of the invention. The objectives and other advantages of the disclosed input device will be realized and attained by the structure particularly pointed out in the written description and claims thereof as well as the appended drawings.

### BRIEF DESCRIPTION OF THE DRAWINGS

The following detailed description of exemplary embodiments can be read in connection with the accompanying drawings in which like numerals designate like elements and in which:

FIG. 1 is a block diagram schematically showing a configuration example of an imaging device according to an exemplary embodiment;

FIG. 2 is a plan view illustrating a configuration example of an image sensor seen from an imaging optical system side.

FIGS. 3A and 3B are diagrams illustrating a configuration example of normal pixels.

FIGS. 4A-4C are diagrams illustrating a configuration example of phase-difference detection pixels.

FIG. 5 is a diagram illustrating examples of a right light-shielding sequence obtained from a line in which right light-shielding pixels exist and a left light-shielding sequence obtained from a line in which left light-shielding pixels paired with the right light-shielding pixels exist.

FIG. 6 is a diagram for describing a relationship between phase-difference and defocus amount.

FIG. 7 is a diagram schematically illustrating a first irradiating light and a second irradiating light emitted by an illumination unit according to an exemplary embodiment.

FIGS. 8A and 8B are diagram schematically illustrating wavelengths of the first irradiating light and the second irradiating light according to an exemplary embodiment.

FIGS. 9A-9D are diagram schematically illustrating phase-difference signals formed by each color light and by white light.

FIG. 10 is a diagram schematically illustrating an usage example in which the imaging device is used in an endoscope system.

FIG. 11 is a partial cross-sectional view of a distal end of an endoscope system of FIG. 10.

FIGS. 12A and 12B are enlarged views illustrating an illumination unit, which has Red LED, Green LED and Blue LED.

FIG. 13 is an exemplary patterns of arranging phase-difference detection pixels.

### DETAILED DESCRIPTION

Exemplary embodiments are explained below with reference to the drawings.

### 1. Embodiments of Imaging Device

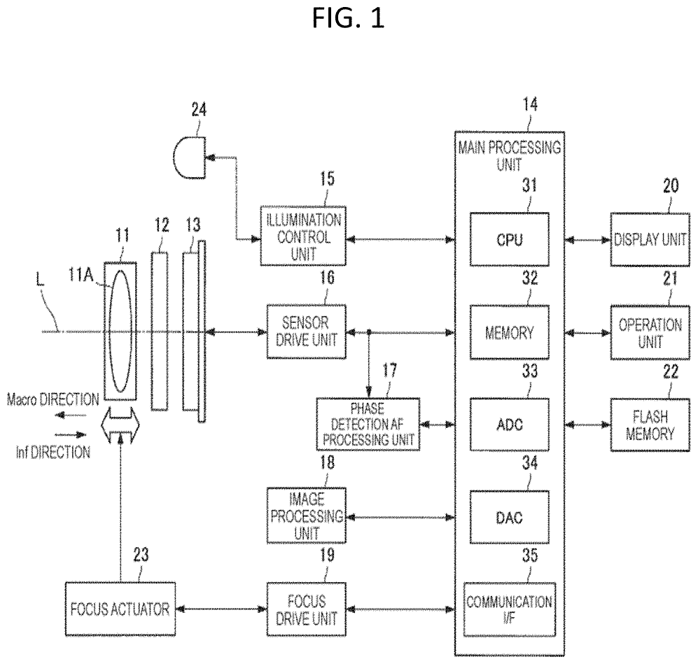

FIG. 1 is a block diagram schematically showing a configuration of an imaging device according to an exemplary embodiment. As shown FIG. 1, an imaging device may include a lens barrel 11, an optical filter 12, an image sensor 13, a main processing unit 14, an illumination control unit 15, a sensor drive unit 16, a PDAF processing unit 17, an image processing unit 18, a focus drive unit 19, a display unit 20, an operation unit 21, a flash memory 22, a focus actuator 23, and an illumination unit 24.

In the exemplary embodiment, the imaging device may be integrally configured with the lens barrel 11. However, the lens barrel 11 may be detachably attached to the imaging device. The lens barrel 11 may include an imaging optical system 11A such as a lens group and a diaphragm and collects light incident thereon on the image sensor 13 via the optical filter 12. The lens group of the imaging optical system 11A has a focus position that is movable in an optical axis L direction, thereby adjusting a focus. The optical filter 12 is configured to reduce false color and moire generated in an image taken by the image sensor 13. Thus, the optical filter 12 may be an optical low-pass filter that attenuates a part of components of light from the imaging optical system 11A and emits the light toward the image sensor 13.

The image sensor 13 is configured to take an image of a subject (not shown) such that light from the subject passes through the imaging optical system 11A and is incident into the image sensor 13 via the optical filter 12. In this embodiment, the image sensor 13 may be a complementary metal oxide semiconductor (CMOS) image sensor, or a charge coupled device (CCD) image sensor. The image sensor 13 supplies image signals to the sensor drive unit 16.

The main processing unit 14 controls each block included in the imaging device. The main processing unit 14 may include a central processing unit (CPU) 31, a memory 32, an analog to digital converter (ADC) 33, a digital to analog converter (DAC) 34, and a communication interface (I/F) 35.

The CPU 31 controls the illumination control unit 15, the flash memory 22, or the like by executing programs stored on the memory 32, thereby causing various kinds of processing to be executed such as AF, taking an image, various kinds of image processing, and recording a taken image.

The memory 32 includes a volatile memory such as a random access memory (RAM), a nonvolatile memory such as an electrically erasable programmable read only memory (EEPROM), or the like and stores the programs to be executed by the CPU 31 and data necessary to operate the CPU 31. The memory 32 may store data regarding an AF parameter for PDAF described below.

The ADC 33 performs AD conversion from an analog signal into a digital signal. The DAC 34 performs DA conversion from a digital signal into an analog signal. The communication I/F 35 controls communication with the Internet or the like.

The illumination control unit 15 performs control so that the illumination unit 24 emits illumination light with which a subject is illuminated as well as auxiliary light for PDAF. In an exemplary embodiment, the illumination control unit 15 causes the illumination unit 24 to emit (turn on) an electronic flash serving as light with which a subject is illuminated, in synchronization with image taking operation of the image sensor 13. Also, the illumination control unit 15 causes the illumination unit 24 to emit torch auxiliary light in synchronization with the PDAF measurement.

The sensor drive unit 16 controls the image sensor 13 to take an image. The sensor drive unit 16 also performs AD conversion of image signals of the image taken by the image sensor 13 as necessary, and supplies the image signals to the main processing unit 14 and the PDAF processing unit 17.

The PDAF processing unit 17 calculates a lens moving amount for moving the lens position of the imaging optical system 11A based on the PDAF. As will be described in detail below, in the PDAF, pixel values of phase-difference detection pixels among the image signals from the sensor drive unit 16 are calculated, and the lens moving amount is supplied to the main processing unit 14.

The image processing unit 18 performs image processing, such as y conversion, color interpolation, and compression/expansion using a predetermined compression/expansion method such as the joint photographic experts group (JPEG), with respect to the image taken by the image sensor 13 and supplied via the sensor drive unit 16 and the main processing unit 14.

The focus drive unit 19 drives the focus actuator 23 in accordance with control by the main processing unit 14 and moves the lens position of the imaging optical system 11A in the optical axis L direction, thereby adjusting a focus.

The display unit 20 may include a liquid crystal display (LCD) panel to display information regarding an image-taking mode of the imaging device, a preview image before taking an image, an image for checking after taking an image, an image in a focused state at the time of AF, and the like. The operation unit 21 is a switch group to be operated by a user and includes a power supply switch, a release (image-taking trigger) switch, a zoom operation switch, an image-taking mode selection switch, and the like. The flash memory 22 is detachable from the imaging device. A taken image supplied from the main processing unit 14 is recorded (stored) on the flash memory 22.

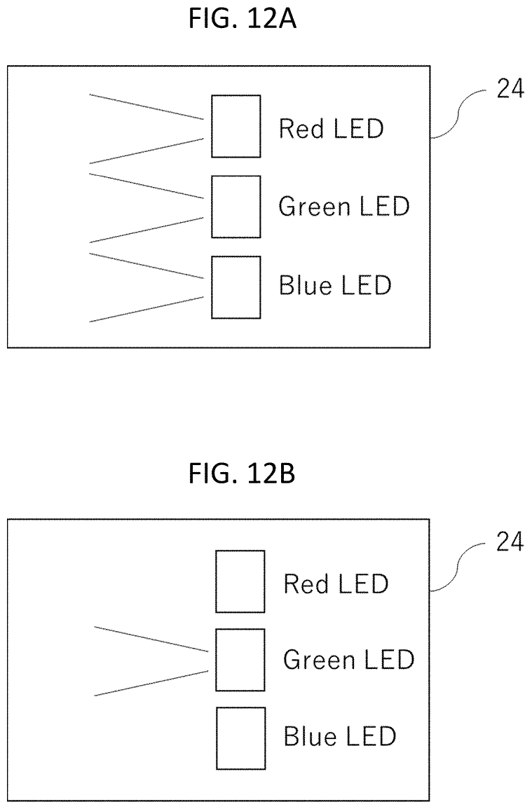

In this exemplary embodiment, the illumination unit 24 emits a first irradiating light with which a subject is illuminated and a second irradiating light for the PDAF in accordance with control by the illumination control unit 15. FIG. 12A and FIG. 12B are enlarged views illustrating the illumination unit 24. The illumination unit 24 has Red LED, Green LED and Blue LED. The first irradiating light is white light. As shown in the FIG. 12A, the illumination unit 24 irradiate the first irradiating light by making Red LED, Green LED and Blue LED emit. The second irradiating light is single color light, which may be one selected from green, blue, red, and any other color light. Thus, the second irradiating light has a wave length range narrower than that of the first irradiating light.

Examples for the illumination unit 24 include, but not limited to, a flash illumination device using a xenon tube, an LED illumination device including a light emitting diode (LED) capable of continuously emitting light, or the like. In a case where the imaging device is mounted on a medical device such as an endoscope, or a portable device such as a smartphone, it is possible to employ a comparatively small LED illumination device as the illumination unit 24.

In this embodiment, the PDAF processing unit 17 is arranged outside the image sensor. Alternatively, the PDAF processing unit 17 may be included in or incorporated into the image sensor 13.

Moreover, the PDAF processing unit 17 may be realized by either hardware or software. In a case where the PDAF processing unit 17 is realized by software, for example, programs included in the software are installed in a computer, such as the main processing unit 14, and are executed by the CPU 31 of the main processing unit 14. In a case where the PDAF processing unit 17 is realized by hardware, for example, a circuit performing the PDAF may be connected to the main processing unit 14 and is controlled by the CPU 31 of the main processing unit 14.

In the exemplary embodiment, the CPU 31 performs in accordance with the programs not necessarily in time series in the order shown as flowcharts described below. That is, the processing that the CPU 31 performs in accordance with the programs also includes processing executed in parallel or individually (for example, parallel processing or processing to be performed by object).

The programs may be recorded in advance on the memory 32 serving as a non-transitory recording medium provided in the main processing unit 14 serving as a computer. Alternatively, the programs may be stored (recorded) on, for example, the flash memory 22 that is a removable recording medium and be provided as so-called packaged software.

The programs may be installed not only in the main processing unit 14 from the flash memory 22 but also in the memory 32 provided therein by being downloaded into the main processing unit 14 via a communication network such as the Internet or a broadcast network such as terrestrial broadcasting.

### 2. Configuration Example of Image Sensor

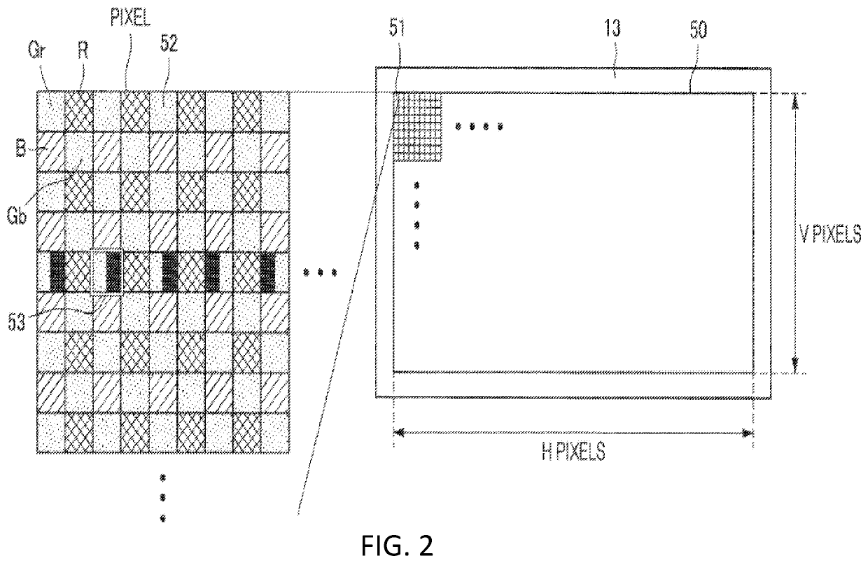

FIG. 2 is a plan views illustrating a configuration example of the image sensor 13 seen from the image optical system 11A side. As shown in FIG. 2, the image sensor 13 may include a light receiving surface 50 that receives light. The light receiving surface 50 includes HxV number of pixels (H represents the number of pixels lined up in a horizontal row and V represents the number of pixels lined up in a vertical column).

In this exemplary embodiment, the image sensor 13 also includes a substrate on which an exposure controlling circuit for controlling a first exposure time for the first irradiating light and a second exposure time for the second irradiating light. Moreover, the image sensor may include the PDAF processing circuit on the substrate of the image sensor 13.

The light receiving surface 50 is divided into, for example, rectangular blocks each of which serves as a pixel group including a plurality of pixels. As shown in FIG. 2, a pixel block 51 includes 9×9 pixels as a pixel group. In the pixel block 51, for example, (primary color) color filters of red (R), green (G), and blue (B) may be provided in a Bayer array on the pixels in an on-chip manner. Under the Bayer array, the color filter pattern is 50% green, 25% red and 25% blue. Alternatives to the Bayer filter may include both various modifications of colors and arrangement and completely different technologies, such as color co-site sampling, the Foveon X3 sensor, the dichroic mirrors or a transparent diffractive-filter array.

In the exemplary embodiment, the pixels on which the color filters of R, G, and B are provided are referred to as an R pixel, a G pixel, and a B pixel, respectively. The R pixel, the G pixel, and the B pixel have spectral sensitivities of R, G, and B light, respectively, because of the on-chip color filters. In the Bayer array, 2×2 pixels (2×2 means horizontal row×vertical column) are considered to be a basic unit, and G pixels are arranged in diagonal positions and an R pixel and a B pixel are arranged in the remaining two positions.

In FIG. 2, in 2×2 pixels considered to be a basic unit, for example, the R pixel may be arranged in an upper right position, the B pixel may be arranged in a lower left position, and the G pixels may be arranged in an upper left position and a lower right position. The G pixel in the upper left position of the basic unit is denoted by Gr, and the G pixel in the lower right position thereof is denoted by Gb. In the Bayer array, the above basic unit is repeatedly arranged horizontally and vertically.

The light receiving surface 50 includes a plurality of phase-difference detection pixels 53 for detecting a phase-difference to be used for the PDAF, and normal pixels (pixels used for the purpose of obtaining an image to serve as a taken image) 52 that are other than the phase-difference detection pixels 53 and are not used to detect a phase-difference.

In the exemplary embodiment, left half portions or right half portions of the phase-difference detection pixels 53 are shielded in order to receive, for example, light passed through a right half portion or left half portion serving as different regions of an exit pupil of the imaging optical system 11A.

### 3. Configuration Example of Pixels

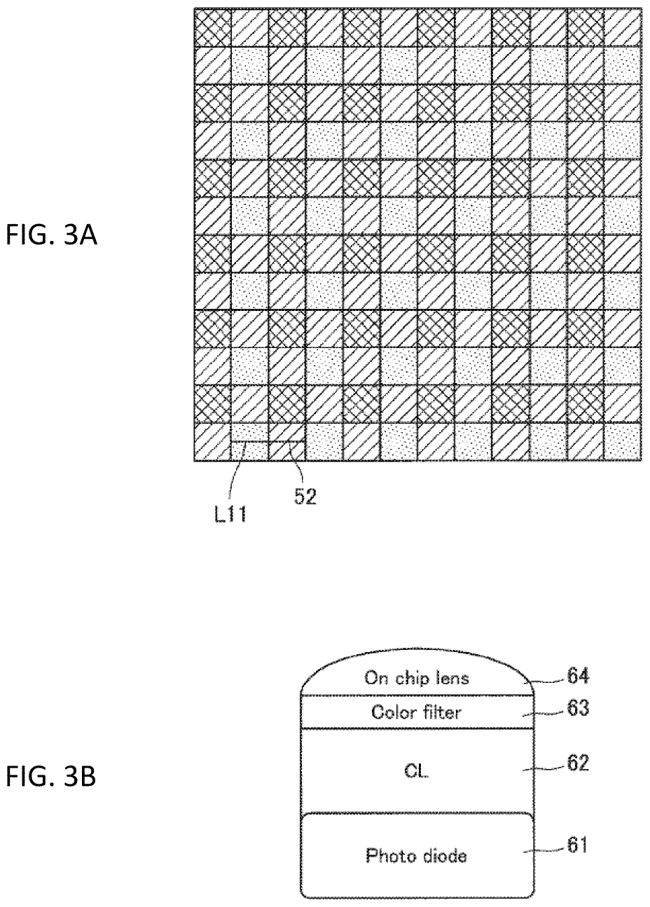

FIGS. 3A and 3B are diagrams illustrating a configuration example of the normal pixels 52. FIG. 3A is a plan view illustrating a configuration example of a region only including the normal pixels 52 in the light receiving surface 50. FIG. 3B is a cross-sectional view schematically illustrating a cross-section of the normal pixel 52 taken along a line segment L11 of FIG. 3A.

The normal pixel 52 is configured so that a photo diode (PD) 61, a contact layer (CL) 62, a color filter 63, and an on-chip lens (microlens) 64 are laminated from the bottom as shown in FIG. 3B. In the normal pixel 52, among light incident on the on-chip lens 64, light having a predetermined color component passes through the color filter 63 and is incident on the PD 61 via the transparent CL 62. In the PD 61, light incident thereon is received and is subjected to photoelectric conversion. An electric signal obtained as a result of the photoelectric conversion in the PD 61 is output as a pixel value of the normal pixel 52.

In an exemplary embodiment, the color filter 63 may be omitted. In this situation, the normal pixels and the phase-difference detection pixels do not have color filters, and thus white light is incident on the PD 61.

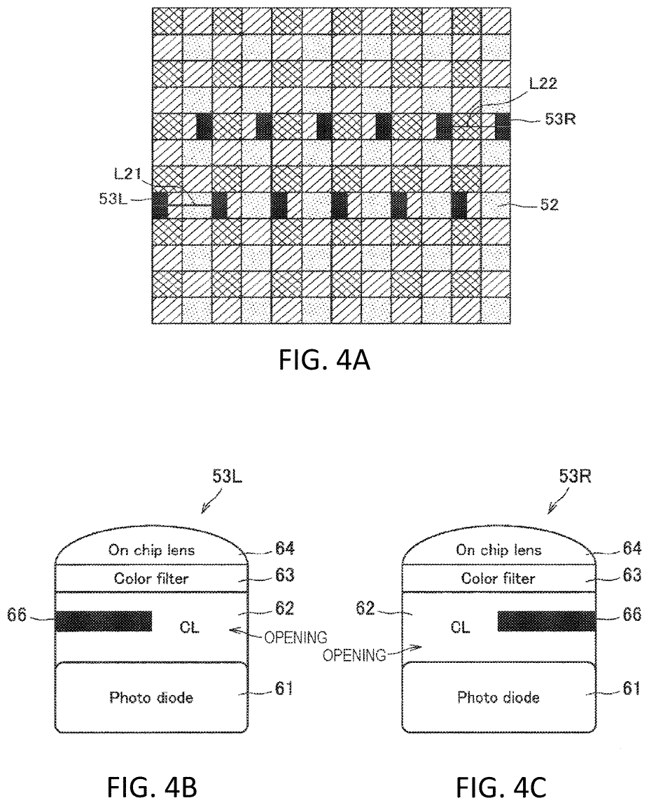

FIG. 4A-4C are the diagrams illustrating a configuration example of phase-difference detection pixels 53. FIG. 4A is a plan view illustrating a configuration example of a region including phase-difference detection pixels 53 in the light receiving surface 50. FIG. 4B is a cross-sectional view schematically illustrating a cross-section of a left light-shielding pixel 53L among phase-difference detection pixels 53 taken along a line segment L21 of FIG. 4A. FIG. 4C is a cross-sectional view schematically illustrating a cross-section of a right light-shielding pixel 53R among phase-difference detection pixels 53 taken along a line segment L22 of FIG. 4A.

As shown in In FIG. 4A, among R pixels, G pixels, and B pixels of the light receiving surface 50, some G pixels serve as phase-difference detection pixels 53. Note that, instead of the G pixels, some R pixels or some B pixels may also be employed as phase-difference detection pixels. The organization in the body cavity mainly reflects red color when the illumination unit 24 irradiate the first irradiating light and the second irradiating light. Thus, R pixels could be employed as phase-difference detection pixels for improving detection of the reflected light from the organization in the body cavity.

Phase-difference detection pixels 53 may include left light-shielding pixels 53L and right light-shielding pixels 53R. As shown in FIG. 4B, the left light-shielding pixel 53L includes a left half portion (layer) 66 configured to shield light in order to, for example, receive light passed through a right half portion serving as a different region of the exit pupil of the imaging optical system 11A. As shown in FIG. 4C, the right light-shielding pixels 53R includes a right half portion (layer) 66 configured to shield light in order to, for example, receive light passed through a left half portion serving as a different region of the exit pupil of the imaging optical system.

In this exemplary embodiment, the left and right half light-shielding layers 66 are made of metal such as Al, Cu, or W metal, but they are not limited to the metal. They may include a metal layer and a non-metal layer.

In order to detect a phase-difference between two images obtained by splitting the exit pupil of the imaging optical system 11A, the left light-shielding pixel 53L and the right light-shielding pixel 53R are paired.

In the exemplary embodiment, the structural elements in phase-difference detection pixel 53, which are configured in the same way as the normal pixel 52 in FIG. 3, are denoted by the reference numerals same as the structural elements in the normal pixel 52. Thus, the same description thereof will be omitted as appropriate.

Phase-difference detection pixel 53 is similar to the normal pixel 52 in that the PD 61 to the on-chip lens 64 are included. However, phase-difference detection pixel 53 is different from the normal pixel 52 in that a light-shielding film 66 is provided in the CL 62.

In the left light-shielding pixel 53L among phase-difference detection pixels 53, as illustrated in FIG. 4B, the light-shielding film 66 is provided to shield light incident on the left half portion of the left light-shielding pixel 53L. With this configuration, in the left light-shielding pixel 53L, only the right half portion from the center of the on-chip lens 64, which is seen from the on-chip lens 64 side, is opened. As a result, for example, light passed through the right half portion of the exit pupil of the imaging optical system 11A (of FIG. 1) is received by the left light-shielding pixel 53L.

In the right light-shielding pixel 53R among phase-difference detection pixels 53, as illustrated in FIG. 4C, the light-shielding film 66 is provided to shield light incident on the right half portion of the right light-shielding pixel 53R. With this configuration, in the right light-shielding pixel 53R, only the left half portion from the center of the on-chip lens 64, which is seen from the on-chip lens 64 side, is opened. As a result, light passed through the left half portion of the exit pupil of the imaging optical system 11A is received by the right light-shielding pixel 53R.

In the exemplary embodiment, the phase-difference detection pixels 53 are regularly arranged over the whole light receiving surface 50 in, for example, the horizontal direction. If the number of phase-difference detection pixels 53 is increased, a phase-difference, more specifically, accuracy of the PDAF, is improved. However, an image quality of a taken image is deteriorated. Therefore, it is possible to determine the number of phase-difference detection pixels 53 and arrangement positions thereof in consideration of a trade-off between accuracy of the PDAF and an image quality of a taken image. Thus, in this exemplary embodiment, the number of phase-difference detection pixels 53 is set at least equal to the number of the normal pixels 52.

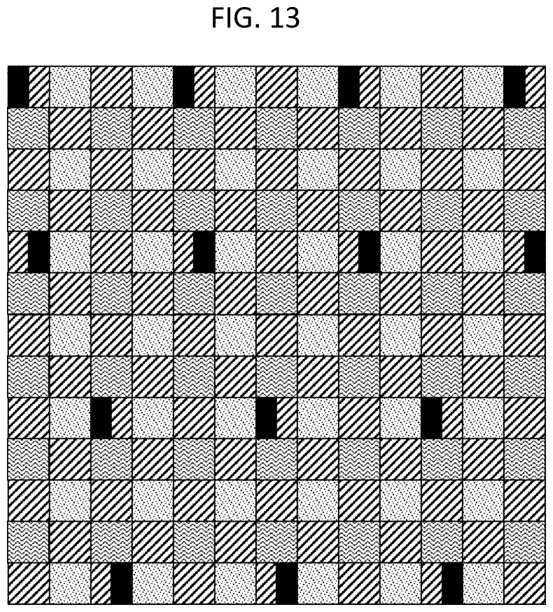

Further, an exemplary arrangement pattern of phase-difference detection pixels 53 may be fixed or different depending on, for example, a position such as a center portion or a peripheral portion of the light receiving surface 50. FIG. 13 is an exemplary patterns of arranging phase-difference detection pixels 53.

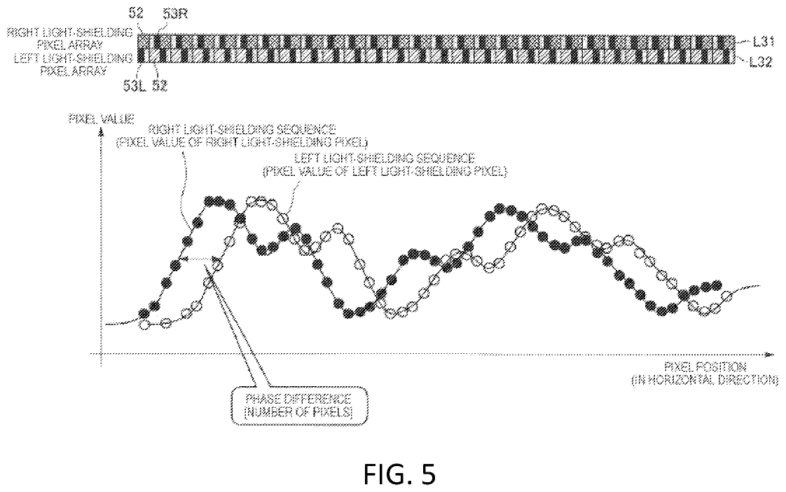

FIG. 5 is a diagram illustrating examples of the right light-shielding sequence obtained from the line in which the right light-shielding pixels 53R exist and the left light-shielding sequence obtained from the line in which the left light-shielding pixels 53L paired with the right light-shielding pixels 53R exist.

In FIG. 5, a horizontal axis shows a position of a pixel and a vertical axis shows a pixel value. As shown in FIG. 5, some G pixels in a line L31 in which R pixels that are the normal pixels 52 exist serve as the right light-shielding pixels 53R. Further, in FIG. 5, some G pixels in a line L32 immediately after the line L31 serve as the left light-shielding pixels 53L. Moreover, for example, the right light-shielding pixels 53R and the left light-shielding pixels 53L that are in lower-left oblique portions from the right light-shielding pixels 53R are paired to detect a phase-difference (between a left light-shielding image and a right light-shielding image).

By using the left light-shielding sequence and the right light-shielding sequence, the phase difference can be obtained (detected) in the unit of the number of pixels. A defocus amount obtained when a subject image is in a focused state is zero, and therefore it is possible to perform AF by moving the lens position of the imaging optical system 11A so that a defocus amount detected on the basis of the phase-difference is zero.

### 4. Description of PDAF

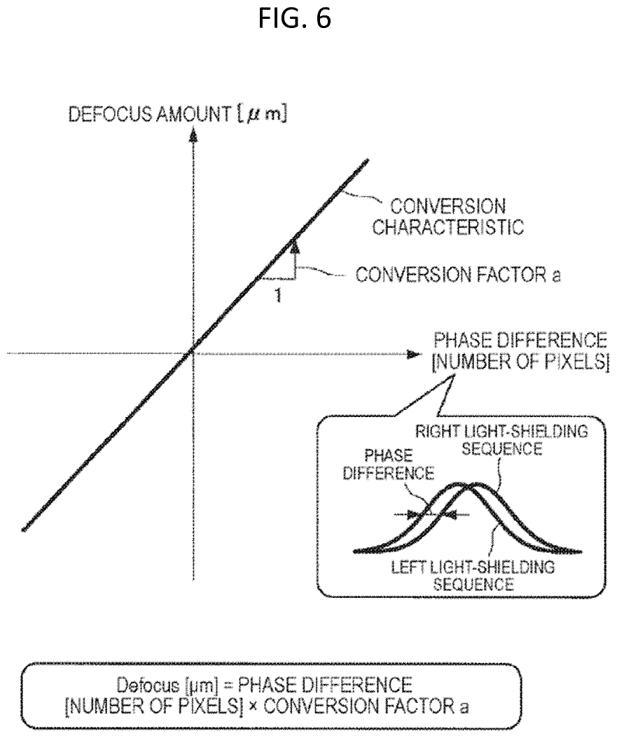

FIG. 6 is a diagram for describing a relationship between the phase-difference and defocus amount. In the PDAF, it is assumed that the phase difference is zero when the lens position is the focusing position, the lens position is, so to speak, directly moved so that the phase difference is close to zero.

Both the phase-difference and the defocus amount indicate a shift amount of a focus of a subject image. However, in AF, the defocus amount is used as a physical amount showing how far it is from a current lens position to the focusing position. That is, in AF, the defocus amount shows a distance and direction from the current lens position to the focusing position.

As shown in FIG. 6, a horizontal axis shows a phase-difference and a vertical axis shows a defocus amount. The phase-difference shows a shift amount of a focus of a subject image as a relative positional relationship between left light-shielding image and right light-shielding image, and a unit thereof is the number of pixels.

The phase-difference and the defocus amount ideally have a linear relationship as illustrated in FIG. 6, and therefore it is possible to obtain one of the phase-difference and the defocus amount on the basis of the other one of the phase-difference and the defocus amount.

Herein, when a coefficient for changing (converting) a phase-difference into a defocus amount is used as a conversion factor a, it is possible to obtain a defocus amount by using a phase-difference in accordance with Expression (1).

Defocus amount[um]=Phase-difference[number of pixels]×Conversion factor a[um/number of pixels] (1)

When the relationship between phase-difference and defocus amount is used as a conversion characteristic, the conversion characteristic is ideally indicated by a straight line. As illustrated in FIG. 6, in a two-dimensional plane in which a horizontal axis shows a phase-difference and a vertical axis shows a defocus amount, the conversion factor a indicates slope of the conversion characteristic indicated by the straight line.

The conversion factor a can be acquired in advance (before shipment) by implementing a test and the like of the imaging device in a factory that manufactures the imaging device.

### 5. Focusing Accuracy of PDAF

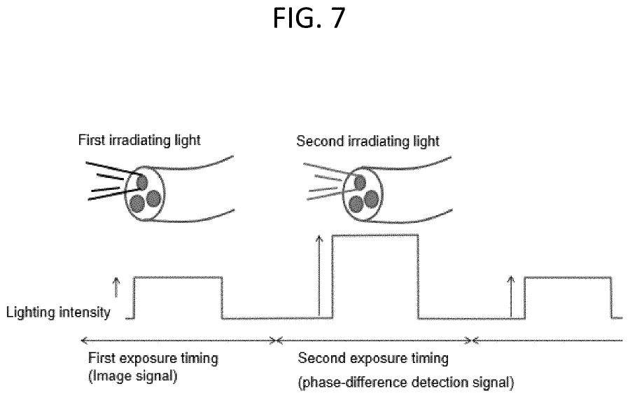

FIG. 7 is a diagram schematically illustrating the illumination unit 24 emitting a first irradiating light and a second irradiating light according to an exemplary embodiment.

In the exemplary embodiment, the illumination control unit 15 controls the illumination unit 24 so that the illumination unit 24 emits the first irradiating light serving as light with which a subject is illuminated, and the illumination unit 24 also emits the second irradiating light serving as an auxiliary light for the PDAF. The first irradiating light may be an electronic flash in synchronization with image taking operation of the image sensor 13. The second irradiating light may be a torch auxiliary light in synchronization with AF operation.

The illumination control unit 15 receives synchronized signals from the exposure control circuit (not shown) that controls a first exposure time for driving the normal pixels 52 to generate an image signal, and a second exposure time for driving the phase-difference detection pixels 53 to generate the phase-difference detection signal. Thus, under the control of the illumination control unit 15, the illumination unit 24 can irradiate the image object with the first irradiating light at the first exposure time, and also irradiate image object with the second irradiating light at the second exposure time. The first exposure time may be the same as or different from the second exposure time. Since each of the phase-difference detection pixels 53 includes a light-shielding layer 66 that covers at least up to 50% of the pixel surface of the phase-difference detection pixel 53 (see FIG. 4), the second exposure time may be set longer than the first exposure time, so that the phase-difference detection pixels are properly exposed.

Alternatively, the exposure control circuit may be arranged on the substrate of the image sensor 13.

Because the light-shielding film 66 covers at least up to 50% of the pixel surface of the phase-difference detection pixels 53, the phase-difference detection pixels 53 have lower sensitivity than the normal pixels 52. For this reason, as shown in FIG. 7, in the exemplary embodiment, the intensity of the second irradiating light is set higher than the intensity of the first irradiating light. For example, the light intensity for the second irradiating light may be set as 200% of the light intensity for the first irradiating light. By this configuration, the phase-difference detection pixels are properly exposed.

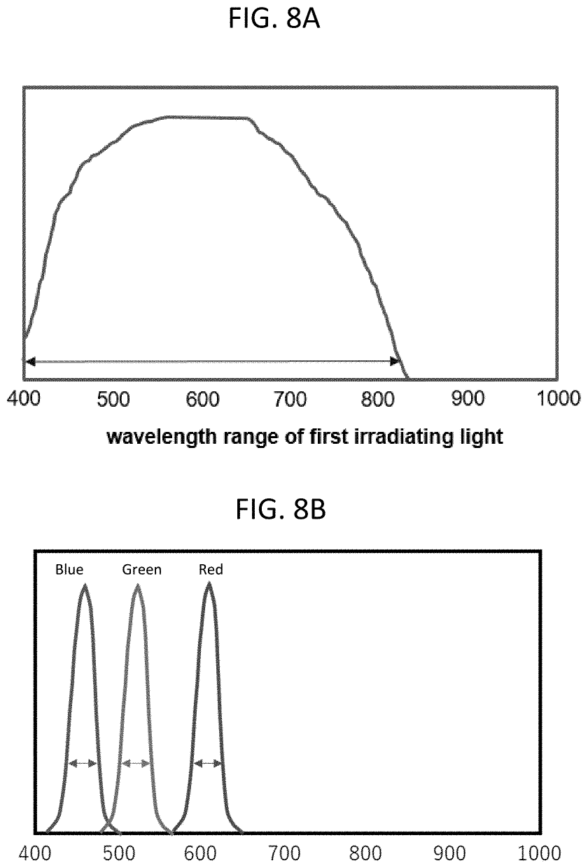

FIGS. 8A and 8B are diagrams schematically illustrating wavelengths of the first irradiating light and the second irradiating light according to an exemplary embodiment. As shown in FIG. 8A, the first irradiating light may be white light, which may have a wavelength range of 400 nm-830 nm. As shown in FIG. 8B, the second irradiating light is a single color light, which may be one of blue color light, green color light, the red color light or any other color light.

In the case that the second irradiating light is the blue color light, a wavelength of the blue color light may include a range of 430 nm to 490 nm, more preferably, a range of 450 nm-470 nm.

In the case that the second irradiating light is the green color light, a wavelength of the green color light may include a range of 500 nm to 560 nm, more preferably, a range of 520 nm-540 nm.

In the case that the second irradiating light is the red color light, a wavelength of the red color light may include a range of 580 nm to 640 nm, more preferably, a range of 600 nm-620 nm.

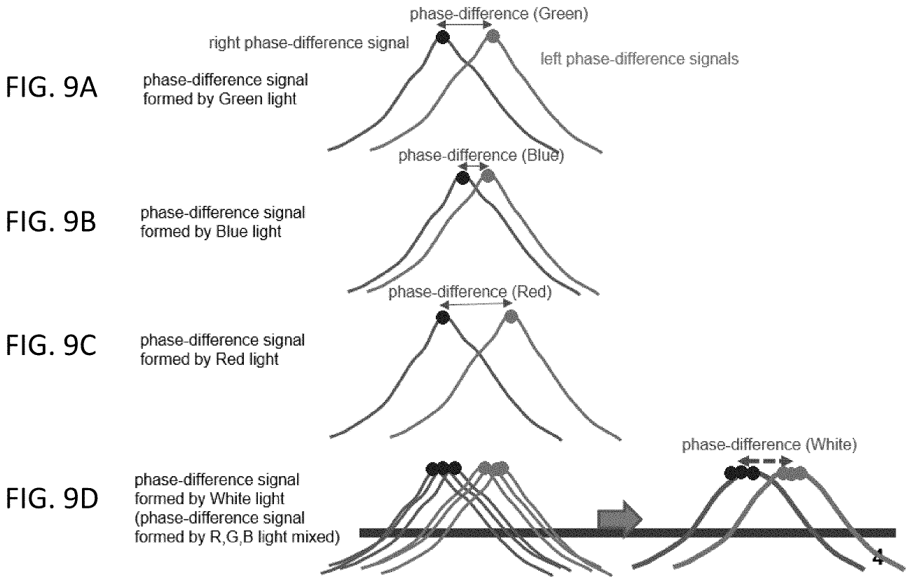

FIGS. 9A-9D are diagrams schematically illustrating phase-difference signals formed by green, blue and red color lights and phase-difference signals formed by white light.

FIG. 9A shows a right phase-difference signal and a left phase-difference signal that are formed by the green light. As shown in FIG. 9A, the right phase-difference signal and the left phase-difference signal have respective definite peak values. The whole shape of the phase-difference signals are acute angle shape. Thus, by measuring the two definite peak values of the right and left phase-difference signals, the value of phase-difference based on the green light can be calculated accurately.

FIG. 9B shows a right phase-difference signal and a left phase-difference signal that are formed by the blue light. As shown in FIG. 9B, the right phase-difference signal and the left phase-difference signal have respective definite peak values. The whole shape of the phase-difference signals are acute angle shape. Thus, by measuring the two definite peak values of the right and left phase-difference signals, the value of phase-difference based on the blue light can be calculated accurately.

FIG. 9C shows a right phase-difference signal and a left phase-difference signal that are formed by the red light. As shown in FIG. 9C, the right phase-difference signal and the left phase-difference signal have respective definite peak values. The whole shape of the phase-difference signals are acute angle shape. Thus, by measuring the two definite peak values of the right and left phase-difference signals, the value of phase-difference based on the red light can be calculated accurately.

However, the values of phase difference calculated by green, blue and red light are different from each other due to the lens not focusing all colors to the same focal plane (chromatic aberration). FIG. 9D shows such a situation, in which right and left phase-difference signals are formed by white light that includes green, blue and red light wavelengths. As shown in FIG. 9D, the right and left phase-difference signals formed by the white light do not have their respective definite peak values because the green, blue and red lights have different peak values. Thus, the whole shape of the phase-difference signals is dull and obtuse angle shaped. As a result, it is difficult to accurately calculate the value of phase difference based on the white light.

### 6. Method of Performing PDAF

Next, a method of performing the PDAF according to an exemplary embodiment will be described.

The imaging device controls the lens position of (the lens group of) the imaging optical system 11A on the basis of the PDAF method. More specifically, the PDAF processing unit 17, which may also be arranged inside the image sensor 13, (is it correct?), performs focus detection on the basis of the PDAF method and calculates a lens moving amount for moving the lens position of (the lens group of) the imaging optical system 11A on the basis of a result of the focus detection.

First, the sensor drive unit 16 uses the exposure control circuit to control the first exposure time for driving the normal pixels 52 to generate the image signal.

The sensor drive unit 16 uses the exposure control circuit to control the second exposure time for driving the phase-difference detection pixels 53 to generate the phase-difference detection signal.

The illumination unit 24 irradiates the image object with the first irradiating light at the first exposure time. In this exemplary embodiment, the first irradiating light is white light.

The illumination unit 24 irradiates the image object with the second irradiating light at the second exposure time. In this exemplary embodiment, the second irradiating light is single color light, which may be one selected from green, blue, red, and any other color light.

Since the first irradiating light is white light, which includes a wave length range of 400 nm-840 nm, the wavelength of the second irradiating light is narrower than the wavelength of the first irradiating light.

As the phase-difference detection pixels 53 have lower sensitivity than the normal pixels 52, the intensity of the second irradiating light is set higher than the intensity of the first irradiating light. For example, the intensity of the second irradiating light may be set 200% of the intensity of the first irradiating light.

Also, the phase-difference detection pixels 53 each have a light-shielding layer 66 that covers at least up to 50% of the pixel surface, the second exposure time may be set longer than the first exposure time so that the phase-difference detection pixels 53 are properly exposed.

### 7. Examples of Imaging Device

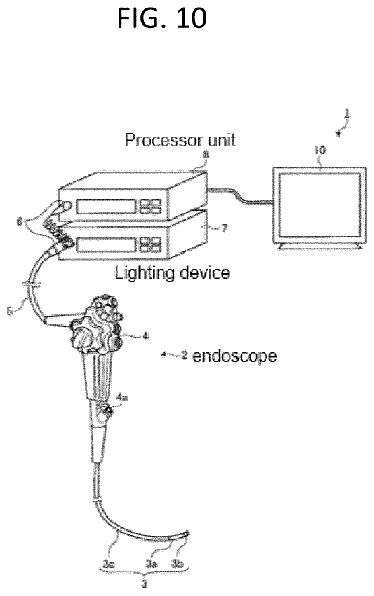

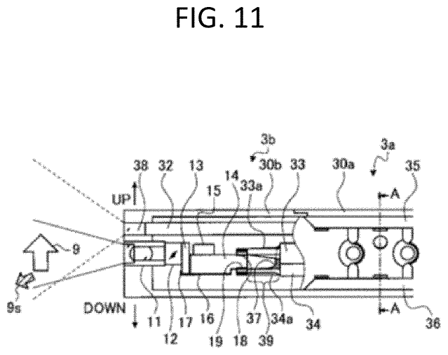

Next, usage examples in which the above-described imaging device is used will be described with reference to FIGS. 10 and 11. FIG. 10 schematically shows an exemplary configuration of an endoscope system 1 that uses the above described imaging device, and FIG. 11 is a partial cross-sectional view of a distal end of an endoscope 2 of FIG. 10.

As shown in FIG. 10, the endoscope system 1 includes an endoscope 2, a universal cord 5, a connector 6, a light source device 7, a processor unit (control device) 8, and a display device 10.

The endoscope 2 captures a subject image by inserting an insertion portion 3 into a body cavity of a subject, and outputs an imaging signal. The universal cord 5 includes a plurality of cables 33, 34 (FIG. 11) extending to a distal end part 3b of the insertion portion 3 of the endoscope 2 and is connected to the imaging device provided at the distal end part 3b of the insertion portion 3. The connector 6 is provided at the proximal end of the universal cord 5 and is connected to the light source device 7 and the processor unit 8.

The light source device 7 irradiates white light via the connector 6 and the universal cord 5, and the white light becomes illumination light irradiated from an illumination window 38 at the distal end part 3b toward a subject. The white light is used for generating image signals. The light source device 7 also irradiates single color light, which may be one of green, blue, red, or any other color light, via the connector 6 and the universal cord 5, and the single color becomes illumination light irradiated from the illumination window 38 toward the subject. The singe color light is used for generating the phase-difference detection signals according to the PDAF mechanism.

The processor unit 8 performs a predetermined image processing on the image signals output from the connector 6 and controls the entire endoscope apparatus 1. The display device 10 displays an image of the subject processed by the processor unit 8.

An operation unit 4 provided with various buttons and knobs for operating the endoscope 2 is connected to the proximal end side of the insertion unit 3 of the endoscope 2. The operation unit 4 includes a treatment instrument insertion port 4a for inserting a treatment instrument such as a bioforceps, an electric knife, an inspection probe, or the like into a body cavity of the subject.

The insertion part 3 includes the distal end part 3b that includes the imaging device, a bendable part 3a that is continuously provided on the proximal end side of the distal end part 3b and is bendable in an up-and-down direction, and a flexible tube part 3c that is continuously provided on the proximal end side of the curved part 3a.

At the distal end part 3b of the endoscope 2, as shown in FIG. 11, a light guide 32 for transmitting the white illumination light and the single light from the light source device 7 is disposed and connected to the illumination window 38. From the illumination window 38, the white illumination light and single color light are irradiated toward the subject.

Also, in the distal end part 3b, the imaging device is provided. The imaging device includes an image sensor 13, which may include an exposure control circuit and a PDAF control unit built in a substrate of the image sensor. The exposure control circuit controls the first exposure time driving the normal pixels 52 of the image sensor to generate the image signal and controls the second exposure time driving the phase-difference pixels 53 to generating the phase-difference detection signals. The PDAF control unit controls the movement of object lens 11 for performing the PDAF based on the calculation of the values of the phase-difference detection signals.

As shown in FIG. 11, an upward direction (UP) corresponds to an upward direction of an image displayed on the display device 10, and a downward direction (DOWN) corresponds to a downward direction of an image displayed on the display device 10.

The imaging device may also include a lens unit 11. The image sensor 13 may be CCD or CMOS image sensor arranged on a proximal end side of the lens unit 11, and adhered to the inside of a tip portion 30b of the distal end part 3b with an adhesive. The lens unit 11 may include a plurality of objective lenses. The imaging device may further include a flexible substrate 16 extending in an optical axis direction from the image sensor 13, a laminated substrate 14 including a plurality of conductive layers formed on the surface of the flexible substrate 16, and a glass lid 12 adhering to the imaging sensor 13 and covering a light-receiving surface of the imaging sensor 13.

An image of an object 9 taken by the lens unit 11 (and an image of a shadow 9s thereof) is detected by the image sensor 13 arranged at an imaging position of the lens unit 11 and is converted into an imaging signal. The imaging signal (output signal) is output to the processor 8 via the flexible substrate 16, the laminated substrate 14, an electronic component 15, and the cable 33 or 34.

The above-described imaging device may be also used for, for example, various electronic devices that senses light such as visible light, infrared light, ultraviolet light, or X-rays, such as a digital camera and a portable appliance with a camera function; an in-vehicle sensor that takes images of the front and the back of a car, surroundings, the inside of the car, and the like, a monitoring camera that monitors travelling vehicles and roads, and a distance sensor that measures distances between vehicles and the like, which are used for safe driving (e.g., automatic stop), recognition of the condition of a driver, and the like; a TV, a refrigerator, and an air conditioner, to takes images of a gesture of a user and operate appliance in accordance with the gesture; a monitoring camera for crime prevention and a camera for personal authentication; skin measurement equipment that takes images of the skin and a microscope that takes images of the scalp; an action camera and a wearable camera for sports and the like; a camera for monitoring the condition of the field and crops.

The present disclosure is not limited to the above described exemplary embodiments. A person skilled in the art may find various alterations and modifications within the scope of the appended claims, and it should be understood that they will naturally come under the technical scope of the present disclosure. Further, the effects described in this specification are merely illustrative or exemplified effects, and are not limitative. That is, with or in the place of the above effects, the technology according to the present disclosure may achieve other effects that are clear to those skilled in the art from the description of this specification.

What is claimed is:

1. An imaging device for performing phase-difference detection auto-focus, comprising;
an image sensor including a plurality of normal pixels and a plurality of phase-difference detection pixels arranged in a matrix shape;
an exposure control circuit controlling a first exposure time driving the normal pixels for generating image signals, and a second exposure time driving the phase-difference detection pixels for generating phase-difference detection signals; and
a lighting device irradiating the imaging object with a first irradiating light at the first exposure time, and irradiating the imaging object with a second irradiating light at the second exposure time,
wherein a wave length range of the second irradiating light is narrower than a wave length range of the first irradiating light.

2. The imaging device according to claim 1, wherein the first irradiating light is white light and the second irradiating light is single color light.

3. The imaging device according to claim 2, wherein the second irradiating light is blue light and includes a wave length range of 430 nm to 490 nm.

4. The imaging device according to claim 2, wherein the second irradiating light is green color light and includes a wave length range of 500 nm to 560 nm.

5. The imaging device according to claim 2, wherein the second irradiating light is red light and includes a wave length range of 580 nm to 640 nm.

6. The imaging device according to claim 2, wherein the second irradiating light is green light and includes a wave length range of 520 nm to 540 nm if the image sensor includes no color filters.

7. The image device according to claim 2, wherein the image sensor includes a photo diode having a peak sensitivity at green light.

8. The imaging device according to claim 1, wherein the second irradiating light is irradiated with intensity that is set higher than that of the first irradiating light.

9. The imaging device according to claim 8, wherein the intensity of the second irradiating light is 200% of the intensity of the first irradiating light.

10. The imaging device according to claim 1, wherein the phase-difference detection pixels each include a light shielding layer that covers at least half of a pixel surface of each of the phase-difference detection pixels.

11. The imaging device according to claim 1, wherein the second exposure time is set longer than the first exposure time so that the phase-difference detection pixels are properly exposed.

12. The imaging device according to claim 1, wherein the number of the phase-difference detection pixels is not more than 50% of the total number of pixels of the imaging sensor.

13. The imaging device according to claim 1, wherein the normal pixels and the phase-difference detection pixels are alternately arranged on the matrix shape.

14. The imaging device according to claim 1, wherein the exposure controlling circuit is disposed on a substrate of the image sensor.

15. A method of performing phase-difference detection auto-focus for an imaging device including an image sensor, comprising:
arranging, in the image sensor, a plurality of normal pixels and a plurality of phase-difference detection pixels in a matrix shape;
controlling, by a exposure controlling circuit arranged the image sensor, a first exposure time driving the normal pixels for generating image signals, and a second exposure time driving the phase-difference detection pixels for generating phase-difference detection signals; and
irradiating, by a lighting source, an object to be imaged with a first irradiating light at the first exposure time, and irradiating the object with a second irradiating light at the second exposure time,
wherein a wave length range of the second irradiating light is narrower than a wave length range of the first irradiating light.

16. The method according to claim 15, wherein the first irradiating light is white light and the second irradiating light is single color light.

17. The method according to claim 16, wherein the second irradiating light is blue light and includes a wave length range of 430 nm to 490 nm.

18. The method according to claim 16, wherein the second irradiating light is green color light and includes a wave length range of 500 nm to 560 nm.

19. The method according to claim 16, wherein the second irradiating light is red light and includes a wave length range of 580 nm to 640 nm.

20. The method according to claim 16, wherein the second irradiating light is green light and includes a wave length range of 520 nm to 540 nm if the image sensor includes no color filters.

21. The method according to claim 15, wherein the second irradiating light is irradiated with intensity that is set higher than that of the first irradiating light.

22. An endoscope system, comprising:
an endoscope including a tip portion to be inserted into an object;
the imaging device according to claim 1, the imaging device arranged inside the tip portion; and
a lighting device connected to the endoscope and providing illumination for the imaging device to capture an image of a region of the object to be examined based on the phase-difference detection auto-focus.

23. The endoscope system according to claim 22, wherein the first irradiating light is white light and the second irradiating light is single color light.

24. A non-transitory computer-readable medium in an electronic device that includes a built-in image sensor, a lighting source, a processor, and memory for storing programs to be executed by the processor, comprising instructions of identifying relevant information of an object, performs a process comprising:
controlling a first exposure time driving normal pixels of the image sensor for generating image signals of an object to be imaged, and a second exposure time driving phase-difference detection pixels of the image sensor for generating phase-difference detection signals; and
irradiating, by the lighting source, the object to be imaged with a first irradiating light at the first exposure time, and irradiating the object with a second irradiating light at the second exposure time,
wherein a wave length range of the second irradiating light is narrower than a wave length range of the first irradiating light.

25. The non-transitory computer-readable medium according to claim 24, wherein the first irradiating light is white light and the second irradiating light is single color light.
